(19) United States
(12) Patent Application Publication
(10) Pub. No.: US 2024/0059800 A1
(43) Pub. Date: Feb. 22, 2024

(54) MODIFIED GUAYULE RESIN PRODUCT AND RELATED PROCESSES

(71) Applicant: Bridgestone Corporation, Chuo-ku (JP)

(72) Inventor: Mark DeDecker, North Canton, OH (US)

(73) Assignee: Bridgestone Corporation, Chuo-ku (JP)

(21) Appl. No.: 18/257,826

(22) PCT Filed: Dec. 16, 2021
(86) PCT No.: PCT/US2021/072971

§ 371 Date: Jun. 15, 2023

Related U.S. Application Data
(60) Provisional application No. 63/127,539, filed on Dec. 18, 2020.

(51) Int. Cl. C08C 19/25; C08L 7/00

(52) U.S. Cl. CPC C08C 19/25; C08L 7/00

(57) ABSTRACT

Disclosed herein are a modified guayule resin product and related processes for preparing the modified guayule resin product. The modified guayule resin product comprises a mixture of argentatins having at least one functional group, wherein the at least one functional group is provided by a functionalizing compound selected from alkoxysilanes, mercaptosilanes and blocked mercaptosilanes. In a second embodiment, a process for preparing a modified guayule resin product is provided whereby a guayule resin component (comprising a mixture of argentatins) is mixed with a functionalizing compound selected from alkoxysilanes, mercaptosilanes and blocked mercaptosilanes to produce a modified guayule resin comprising functionalized argentatins having at least one functional group provided by the functionalizing compound.

### FIELD

The present application is directed to a modified guayule resin product and related processes for preparing the modified guayule resin product.

### BACKGROUND

The guayule plant (Parthenium argentatum) is a woody shrub-like plant that produces rubber and resin. Processes which are directed to isolating rubber from the guayule plant will produce a considerable amount of guayule resin as a by-product.

### SUMMARY

Disclosed herein are a modified guayule resin product and related processes for preparing the modified guayule resin product.

In a first embodiment, a modified guayule resin product is provided. The modified guayule resin product comprises a mixture of argentatins having at least one functional group, wherein the at least one functional group is provided by a functionalizing compound selected from alkoxysilanes, mercaptosilanes and blocked mercaptosilanes.

In a second embodiment, a process for preparing a modified guayule resin product is provided. According to the process of the second embodiment, a guayule resin component is provided which comprises a mixture of argentatins having —OH groups. The guayule resin component is mixed with a functionalizing compound selected from alkoxysilanes, mercaptosilanes and blocked mercaptosilanes to produce a modified guayule resin comprising functionalized argentatins having at least one functional group provided by the functionalizing compound.

### DETAILED DESCRIPTION

Disclosed herein are a modified guayule resin product and related processes for preparing the modified guayule resin product.

In a first embodiment, a modified guayule resin product is provided. The modified guayule resin product comprises a mixture of argentatins having at least one functional group, wherein the at least one functional group is provided by a functionalizing compound selected from alkoxysilanes, mercaptosilanes and blocked mercaptosilanes.

In a second embodiment, a process for preparing a modified guayule resin product is provided. According to the process of the second embodiment, a guayule resin component is provided which comprises a mixture of argentatins having —OH groups. The guayule resin component is mixed with a functionalizing compound selected from alkoxysilanes, mercaptosilanes and blocked mercaptosilanes to produce a modified guayule resin comprising functionalized argentatins having at least one functional group provided by the functionalizing compound.

### Definitions

The terminology as set forth herein is for description of the embodiments only and should not be construed as limiting the invention as a whole.

As used herein, the term “BR” or “polybutadiene” refers to homopolymer of 1,3-butadiene.

As used herein, the term “majority” refers to more than 50% (e.g., at least 50.1%, at least 50.5%, at least 51%, etc.).

As used herein, the term “minority” refers to less than 50% (e.g., no more than 49.5%, no more than 49%, etc.).

As used herein, the abbreviation Mn is used for number average molecular weight.

As used herein, the abbreviation Mp is used for peak molecular weight.

As used herein, the abbreviation Mw is used for weight average molecular weight.

Unless otherwise indicated herein, the term “Mooney viscosity” refers to the Mooney viscosity, ML₁₊₄. As those of skill in the art will understand, a rubber composition's Mooney viscosity is measured prior to vulcanization or curing.

As used herein, the term “natural rubber” means naturally occurring rubber such as can be harvested from sources such as Hevea rubber trees and non-Hevea sources (e.g., guayule plant and dandelions such as TKS). In other words, the term “natural rubber” should be construed so as to exclude synthetic polyisoprene.

As used herein, the term “guayule rubber” is a sub-category of natural rubber which has been harvested from the guayule plant. In contrast, natural rubber which has not been harvested from the guayule plant is referred to herein as “non-guayule natural rubber” and can include Hevea rubber as well as other sources such as dandelion.

As used herein, the term “phr” means parts per one hundred parts rubber. The one hundred parts rubber is also referred to herein as 100 parts of an rubber component.

As used herein the term “polyisoprene” means synthetic polyisoprene. In other words, the term is used to indicate a polymer that is manufactured from isoprene monomers, and should not be construed as including naturally occurring rubber (e.g., Hevea natural rubber, guayule-sourced natural rubber, or dandelion-sourced natural rubber). However, the term polyisoprene should be construed as including polyisoprenes manufactured from natural sources of isoprene monomer.

As used herein the term “SBR” means styrene-butadiene copolymer rubber.

As used herein, the term “tread,” refers to the portion of a tire that comes into contact with the road under normal inflation and load and the term “subtread” refers to the portion underlying the tread which does not generally come into contact with the road.

Modified Guayule Resin Product

As discussed above, the first embodiment disclosed herein is directed to a modified guayule resin product and the second embodiment disclosed herein provides a process for preparing a modified guayule resin product. According to the first and second embodiments, the modified guayule resin product comprises a mixture of argentatins having at least one functional group, wherein the at least one functional group is provided by a functionalizing compound selected from alkoxysilanes, mercaptosilanes, and blocked mercaptosilanes. As those of skill in the art will understand, argentatins constitute a class of compounds which naturally occur in guayule resin and which contain (in unmodified or non-functionalized form) one or more —OH groups. These argentatins have been categorized into various categories including Argentatin A, Argentatin B, Argentatin C, Argentatin D, Argentatin E, and Argentatin F. The modification or functionalization of the argentatins takes place through bonding of the oxygen atom from an —OH group of an argentatin compound to the silicon atom of a functionalizing compound. In other words, the point of attachment between the argentatin compound and the functionalizing compound is via the oxygen atom from an —OH group of the argentatin compound to the silicon atom of the functionalizing compound. As a non-limiting example, use of a trialkoxysilane for the functioning compound would result in the following modified or functionalized argentatin structure:

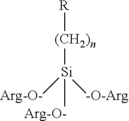

where Arg-O refers to an argentatin compound without the hydrogen atom from its —OH group. Since a trialkoxysilane compound contains three alkoxygroups, it provides for three points of attachment for argentatin compounds. For argentatin compounds which contain more than one —OH group, more than one point of attachment to the silicon atom of an alkoxysilane compound is possible. Overall, the bonding between the argentatins and alkoxysilane compounds (or more broadly, a functionalizing compound) results in a modified guayule resin product.

According to the first and second embodiments, the particular functionalizing compound used to functionalize the argentatins may vary. As discussed above, according to the first and second embodiments, the functionalizing compound is selected from alkoxysilanes, mercaptosilanes, and blocked mercaptosilanes, each as discussed in more detail below.

According to the first and second embodiments, the percentage by mass or weight of the hydrogen atoms from the —OH groups of the argentatins that are replaced by a functional group from the functionalizing compound may vary. In preferred embodiments of the first and second embodiments, the mixture of functionalized argentatins in the modified guayule resin product have at least 50% by mass (e.g., 50%, 55%, 60%, 65%, 70%, 75%, 80%, 85%, 90%, or more) of their hydrogen atoms from their —OH groups replaced with the at least one functional group, preferably 60 to 90% (e.g., 60, 65, 70, 75, 80, 85, or 90%) by mass of their hydrogen atoms from their —OH groups replaced with the at least one functional group. The percentage by mass of hydrogen atoms from the —OH groups of the mixture of argentatins that is replaced by at least one functional group from an alkoxysilane can be considered to be the percentage of functionalization of the mixture of argentatins. In other words, a mixture of argentatins wherein 75% by mass of hydrogen atoms from their —OH groups have been replaced with a functional group from the functionalizing can be considered to be 75% functionalized.

According to the first and second embodiments disclosed herein, the properties (e.g., Tg, Mn and Mw) of the modified guayule resin product may vary. In certain embodiments of the first and second embodiments, the modified guayule resin product has a Tg of −70 to 100° C. (e.g., −70, −60, −50, −40, −30, −20, −10, 0, 10, 20, 30, 40, 50, 60, 70, 80, 90 or 100° C. In certain embodiments of the first and second embodiments, the modified guayule resin product has a Tg within the lower portion of the foregoing range, e.g., a Tg of −60 to 0° C. (e.g., −60, −55, −50, −45, −40, −35, −30, −25, −20, −15, −10, −5 or 0° C.). In other embodiments of the first and second embodiments, the modified guayule resin product has a Tg within the upper portion of the foregoing range of −70 to 100° C., e.g., a Tg of 1 to 100° C. (e.g., 1, 5, 10, 15, 20, 25, 30, 35, 40, 45, 50, 55, 60, 65, 70, 75, 80, 85, 90, 95 or 100° C.), preferably 30 to 90° C. (e.g., 30, 35, 40, 45, 50, 55, 60, 65, 70, 75, 80, 85, 90° C.), more preferably 40 to 90° C. (e.g., 40, 45, 50, 55, 60, 65, 70, 75, 80, 85, 90° C.); modified guayule resin product having a Tg within one of the foregoing ranges may be particularly useful in tire rubber compositions, e.g., for tread compounds. In certain embodiments of the first and second embodiments, the modified guayule resin product has a Mn of 1600 to 5000 grams/mole (e.g., 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900 or 5000 grams/mole), including 2000 to 4500 grams/mole and 2500 to 4000 grams/mole. In certain embodiments of the first and second embodiments, the modified guayule resin product has a Mw of 2000 to 10,000 grams/mole (e.g., 2000, 2500, 3000, 3500, 4000, 4500, 5000, 5500, 6000, 6500, 7000, 7500, 8000, 8500, 9000, 9500, or 10,000 grams/mole), including 4000 to 9000 grams/mole and 5000 to 8000 grams/mole. In certain embodiments of the first and second embodiments, the modified guayule resin has Mn and Mw within the foregoing ranges; in certain such embodiments, the modified guayule resin also has a Tg within one of the foregoing ranges. The Mw and Mn values can be determined by GPC, e.g., using the method described in the working Examples. The Tg values can be determined by DSC, using an instrument such that as manufactured by TA Instruments (New Castle, Delaware), where the measurement is conducted using a temperature elevation of 10° C./minute after cooling at −120° C. Thereafter, a tangent is drawn to the base lines before and after the jump of the DSC curve. The temperature on the DSC curve (read at the point corresponding to the middle of the two contact points) can be used as Tg.

According to the first and second embodiments, the amount of guayule rubber that is present in the modified guayule resin product may vary. As those of skill in the art will understanding, the amount of guayule rubber that is present in the modified guayule resin product will often be dependent upon the amount of guayule rubber that was present in the (unmodified) guayule resin component from which the modified guayule resin product is prepared. However, the amount of guayule rubber present in the modified guayule resin product can also refer to guayule rubber that is added after functionalization of the guayule resin component. In certain embodiments of the first and second embodiments, the modified guayule resin product contains less than 10% by weight (e.g., 9.5%, 9%, 8.5%, 8%, 7.5%, 7%, 6.5%, 6%, 5.5%, 5%, 4.5%, 4%, 3.5%, 3%, 2.5%, 2%, 1.5%, 1%, 0.5%, or less), preferably less than 5% by weight (e.g., 4.5%, 4%, 3.5%, 3%, 2.5%, 2%, 1.5%, 1%, 0.5%, or less) or even less than 1% by weight of guayule rubber.

According to the first and second embodiments disclosed herein, the percentage by weight of functionalized argentatins in the modified guayule resin product may vary. Preferably, according to the first and second embodiments, the mixture of functionalized argentatins constitutes a primary component by weight of the modified guayule resin product. By primary component is meant that of the different components present in the modified guayule resin product (e.g., unmodified argentatins, triglycerides, fatty acids), the component that is present in the largest amount is the functionalized argentatin component (which will generally be a mixture of functionalized argentatins). In more preferred embodiments of the first and second embodiments, the mixture of functionalized argentatins constitutes at least 40% by weight of the modified guayule resin product (e.g., 40%, 50%, 60%, 70%, 80%, 90%, 95% or more), more preferably at least a majority by weight of the modified guayule resin product (e.g., 51%, 55%, 60%, 65%, 70%, 75%, 80%, 85%, 90%, 95% or more), even more preferably at least 60% by weight of the modified guayule resin product (e.g., 60%, 65%, 70%, 75%, 80%, 85%, 90%, 95% or more) or 60-90% by weight (e.g., 60%, 65%, 70%, 75%, 80%, 85%, 90%) of the modified guayule resin product. The amount of functionalized argentatins in the modified guayule resin product can be determined by GPC (e.g., using the procedure described in the working Examples) where the functionalized argentatins will generally be represented by at least the first eluting peak and in many instances at least a portion of the second eluting peak (the functionalized argentatins will generally have a Mn in the range of 1600 to 5000 grams/mole, as discussed infra, and, thus, one of skill in the art will understand how to interpret the GPC results to correspond to the amount of product within this Mn range.) The modified guayule resin product may also contain some portion of triglycerides, fatty acids, and unmodified argentatins and guayules (with the relative amounts varying generally depending upon the amount present in the unmodified guayule resin component and/or any purification processes conducted on the modified guayule resin product to remove such materials.)

Guayule Resin Product

As discussed above, the process of the second embodiment includes use of a guayule resin product which is mixed with a functionalizing compound to produce the modified guayule resin product. Similarly, the modified guayule resin product of the first embodiment can be understood as being a functionalized version of a guayule resin product wherein the functionalizing compound discussed herein is used. As mentioned above, the guayule resin product comprises (includes) a mixture of argentatins which have —OH groups. Preferably, according to the first and second embodiments, the mixture of argentatins constitutes a primary component by weight of the guayule resin component. By primary component is meant that of the different components present in the guayule resin component (e.g., argentatins, triglycerides, fatty acids, and low molecular weight rubber), the component that is present in the largest amount is the argentatin component (which will generally be a mixture of argentatins). In more preferred embodiments of the first and second embodiments, the mixture of argentatins constitutes at least 40% by weight of the guayule resin component (e.g., 40%, 50%, 60%, 70%, 80%, 90%, 95% or more), more preferably at least a majority by weight of the guayule resin component (e.g., 51%, 55%, 60%, 65%, 70%, 75%, 80%, 85%, 90%, 95% or more), even more preferably at least 60% by weight of the guayule resin component (e.g., 60%, 65%, 70%, 75%, 80%, 85%, 90%, 95% or more) or 60-90% by weight of the guayule resin component. Generally, due to the difficulty in separating the argentatin component of guayule resin from other naturally occurring components, the percentage by weight of the guayule resin component which constitutes argentatins will be less than 100%.

According to the first and second embodiments, the amount of guayule rubber that is present in the guayule resin component may vary. In certain embodiments of the first and second embodiments, the guayule resin component contains less than 10% by weight (e.g., 9.5%, 9%, 8.5%, 8%, 7.5%, 7%, 6.5%, 6%, 5.5%, 5%, 4.5%, 4%, 3.5%, 3%, 2.5%, 2%, 1.5%, 1%, 0.5%, or less), preferably less than 5% by weight (e.g., 4.5%, 4%, 3.5%, 3%, 2.5%, 2%, 1.5%, 1%, 0.5%, or less) or even less than 1% by weight of guayule rubber.

Functionalizing Compound

As discussed above, according to the first and second embodiments, the functionalizing compound is selected from alkoxysilanes, mercaptosilanes, and blocked mercaptosilanes. As discussed in more detail below, the alkoxysilanes should be understood to include both sulfur-containing alkoxysilanes as well as non-sulfur-containing alkoxysilanes.

In certain embodiments of the first and second embodiments disclosed herein, the functionalizing compound is a non-sulfur containing alkoxysilane. Preferably according to such embodiments, the non-sulfur containing alkoxysilane has formula (I):

R¹₍₄₋ₙ₎−Si(OR²)ₙ (I)

where n=2, 3, or 4; each R¹ is independently selected from a hydrocarbyl group having 1-20 carbons, preferably 2-18 carbons; and R² is an alkyl group having 1-10 carbons, preferably 1-6, more preferably 1-3 carbons or an aromatic group having 6-18 carbons, preferably 6-12 carbons. When n=2, the non-sulfur containing alkoxysilane can be understood as being a dialkoxysilane. When n=3, the non-sulfur containing alkoxysilane can be understood as being a trialkoxysilane. When n=4, the non-sulfur containing alkoxysilane can be understood as being a tetraalkoxysilane.

In certain embodiments of the first and second embodiments, the functionalizing compound is a non-sulfur containing alkoxysilane silane having formula (I) where n=2, i.e., a dialkoxysilane. Non-limiting examples of non-sulfur containing alkoxysilanes which are dialkoxysilanes include, but are not limited to, dimethyl diimethoxysilane, dimethyl diiethoxysilane, dimethyl dipropoxysilane, dimethyl diisopropoxysilane, diethyl dimethoxysilane, diethyl diethoxysilane, diethyl dipropoxysilane, diethyl diisopropoxysilane, dipropyl dimethoxysilane, dipropyl diethoxysilane, dibutyl dimethoxysilane, dibutyl diiethoxysilane, dipentyl dimethoxysilane, dipentyl diethoxysilane, dihexyl dimethoxysilane, dihexyl diethoxysilane, diheptyl dimethoxysilane, diheptyl diethoxysilane, dioctyl dimethoxysilane, dioctyl diethoxysilane, dinonyl dimethoxysilane, dinonyl diethoxysilane, didecyl dimethoxysilane, didecyl diethoxysilane, diundecyl dimethoxysilane, diundecyl diethoxysilane, didodecyl dimethoxysilane, didodecyl diethoxysilane, ditridecyl dimethoxysilane, ditridecyl diethoxysilane, ditetradecyl dimethoxysilane, dipentadecyl dimethoxysilane, ditetradecyl diethoxysilane, dipentadecyl diethoxysilane, dihexadecyl dimethoxysilane, dihexadecyl diethoxysilane, diheptadecyl dimethoxysilane, diheptadecyl diethoxysilane, dioctadecyl dimethoxysilane, dioctadecyl diethoxysilane, dinonadecyl dimethoxysilane, dinonadecyl diethoxysilane, diphenyl dimethoxysilane, diphenyl diethoxysilane, diphenyl dipropoxysilane, diphenyl diisopropoxysilane, dibenzyl dimethoxysilane, dibenzyl diethoxysilane.

In certain embodiments of the first and second embodiments, the functionalizing compound is a non-sulfur containing alkoxysilane silane having formula (I) where n=3, i.e., a trialkoxysilane. Non-limiting examples of non-sulfur containing alkoxysilanes which are trialkoxysilanes include, but are not limited to, methyl trimethoxysilane, methyl triethoxysilane, methyl tripropoxysilane, methyl triisopropoxysilane, ethyl trimethoxysilane, ethyl triethoxysilane, ethyl tripropoxysilane, ethyl triisopropoxysilane, propyl trimethoxysilane, propyl triethoxysilane, butyl trimethoxysilane, butyl triethoxysilane, pentyl trimethoxysilane, pentyl triethoxysilane, hexyl trimethoxysilane, hexyl triethoxysilane, heptyl trimethoxysilane, heptyl triethoxysilane, octyl trimethoxysilane, octyl triethoxysilane, nonyl trimethoxysilane, nonyl triethoxysilane, decyl trimethoxysilane, decyl triethoxysilane, undecyl trimethoxysilane, undecyl triethoxysilane, dodecyl trimethoxysilane, dodecyl triethoxysilane, tridecyl trimethoxysilane, tridecyl triethoxysilane, tetradecyl trimethoxysilane, tetradecyl triethoxysilane, pentadecyl trimethoxysilane, pentadecyl triethoxysilane, hexadecyl trimethoxysilane, hexadecyl triethoxysilane, heptadecyl trimethoxysilane, heptadecyl triethoxysilane, octadecyl trimethoxysilane, octadecyl triethoxysilane, nonadecyl trimethoxysilane, nonadecyl triethoxysilane, phenyl trimethoxysilane, phenyl triethoxysilane, phenyl tripropoxysilane, phenyl triisopropoxysilane, benzyl trimethoxysilane, benzyl triethoxysilane.

In certain embodiments of the first and second embodiments, the functionalizing compound is a non-sulfur containing alkoxysilane silane having formula (I) where n=4, i.e., a tetraalkoxysilane. Non-limiting examples of non-sulfur containing alkoxysilanes which are tetraalkoxysilanes include, but are not limited to, tetramethoxysilane, tetraethoxysilane, tetra propoxysilane, tetra-isopropoxysilane, tetra butoxysiane, and tetra-isobutoxysilane.

In certain embodiments of the first and second embodiments disclosed herein, the functionalizing compound is a non-sulfur containing bis-alkoxysilane. A non-sulfur containing bis-alkoxysilane can be understood as containing two silicon atoms, preferably separated by a divalent hydrocarbyl group, with each silicon atom having two or three alkoxygroups. In certain embodiments of the first and second embodiments, the non-sulfur containing bis-alkoxysilane has the formula (Y)G(Z) wherein G is a separating group selected from the group consisting of C₁-C₅₀ straight chain and branched alkylene, C₂-C₅₀ straight chain and branched alkenylene, C₆-C₅₀ aromatics, each optionally containing a heteroatom selected from the group consisting of one or more 0, or one or more N, and combinations thereof; and Y and Z can be the same or different and each independently comprise a group of the formula Si(R⁷)ₚ(OR⁸)₃₋ₚ wherein each R⁷ independently comprises C₁-C₂₀ aliphatic, cycloaliphatic or aromatic, R⁸ is C₁-C₆ aliphatic or cycloaliphatic and p is an integer of 0 or 1. In preferred embodiments of the first and second embodiments, when the non-sulfur containing bis-alkoxysilane has the foregoing formula, the G of the non-elastomer reactive filler reinforcing agent is selected from the group consisting of C₂-C₂₀ alkylene, and alkenylene, and C₆-C₂₀ aromatics, each optionally containing a heteroatom selected from the group consisting clone or more O or one or more N, and combinations thereof. In certain embodiments of the first and second embodiments, the non-sulfur containing bis-alkoxysilane has the formula (y)GM and G is selected from the group consisting of C₆-C₂₀ alkylene and alkenylene and each R⁸ is selected from the group consisting of C₁ to C₆ straight-chain and branched aliphatic. In certain embodiments of the first and second embodiments, the non-sulfur containing bis-alkoxysilane has the formula (Y)G(2) and G selected from the group consisting of C₄-C₂₀ straight-chain and branched alkylene and C₄-C₂ straight-chain and branched alkenylene either optionally containing additional carbon atoms in the form of one or more aromatic rings. In particular embodiments, the non-sulfur containing bisalkoxysilane is a bis(trialkoxy)silane with the carbon portion of the alkoxy selected from the group consisting of C₁ to C₆ methyl to hexyl), preferably C₁ to C₁ and even more preferably C₁ to C₇, Specific examples of such bis(trialkoxy)silanes include, but are not limited to, bis(trimethoxysilyl)ethane, bis(triethoxysilyi)ethane, bis(tributoxysilyl)ethane, bis(triethoxysilyl)propane, bis(trirnethoxysilyl)propane, bis(tributoxysilyl)propane, bis(triethoxysilyl)butane, bis(trimethoxysilyl)butane, bis(tributoxysilyl)butane, bis(triethoxysilypisobutane, bis(trimetboxysilyl)isobutane, bis(tributoxysilyl)isobutane, bis(triethoxysilyl)hexane, bis(trirnethoxysilyphexane, bis(tributoxysilyphexane, bis(triethoxysilyl)cyclohexane, bis(trimethoxysilyl)cyclohexane, bis(tributoxysilyi)cyclohexane, bis(trimethoxysilypheptane, bis(triethoxysilyl)heptane, bis(tributoxysilypheptane, bis(triethoxysilyl)octane, bis(trimethoxysilyi)octane, bis(tributoxysilyl)octane, bis(triethoxysilyl)nonane, bis(trirnethoxysilyl)nonane, bis(tributoxysilyl)nonane, bis(triethoxysilyl)decane, bis(trimethoxysilyi)decane, bis(tributoxysilyl)decane, bis(triethoxysilyl)dodecane, bis(trirnethoxysilyl)dodecane, bis(tributoxysilyl)dodecane, bisftriethoxysilyptetraderane, bis(trimethoxysilyl)tetradecane, bis(tributoxysilyptetraderane, bisftriethoxysilypoctadecane, bis(trimetboxysilyl)octadecane, bis(tributoxysilypoctadecane, and mixtures thereof.

In certain embodiments of the first and second embodiments disclosed herein, the functionalizing compound is a sulfur-containing alkoxysilane having 2-6 alkoxysilane groups. Preferably according to such embodiments, the sulfur-containing alkoxysilane is selected from disulfide alkoxysilanes or tetrasulfide alkoxysilanes. A disulfide alkoxysilane can be understood as having two sulfur atoms (single bonded to each other), each sulfur of which is bonded to a separating alkylene group that is bonded to a silicon atom that is in turn has two or three alkoxygroups. A tetrasulfide alkoxysilane can be understood as having four sulfur atoms (single bonded to each other), with the end sulfurs each bonded to a separating alkylene group that is bonded to a silicon atom that in turn has two or three alkoxygroups. In certain embodiments of the first and second embodiments, the disulfide alkoxysilane has the formula (alkoxy) a (alkyl)₃₋ₐSi—(CH₂)_{b}—S—S—(CH₂)_{b}—Si(alkyl)₃₋ₐ(alkoxy)ₐ where a is 2 or 3; b is an integer of 1 to 10, preferably 2 to 8, more preferably 2 or 3; and the alkyl in the alkoxy groups is selected from alkyl of 1-10 carbons, preferably 1 to 6 carbons, more preferably 1 to 4 carbons. In certain embodiments of the first and second embodiments, the tetrasulfide alkoxysilane has the formula (alkoxy)_{d}(alkyl)_{3-d}Si—(CH₂)ₑS—S—S—S—(CH₂)ₑ—Si(alkyl)_{3-d}(alkoxy)_{d} where d is 2 or 3; e is an integer of 1 to 10, preferably 2 to 8, more preferably 2 or 3; and the alkyl in the alkoxy groups is selected from alkyl of 1-10 carbons, preferably 1 to 6 carbons, more preferably 1 to 4 carbons. Alternatively, in other embodiments, the tetrasulfide alkoxysilane has the alkoxysilane alkylene moiety on only one end of the sulfur chain (e.g., the first sulfur) and at the other end of the sulfur chain (e.g., the fourth sulfur), another moiety is present (e.g., thiocarbamoyl, benzothiazole).

In certain embodiments of the first and second embodiments disclosed herein, when the functionalizing compound is a disulfide alkoxysilane it is selected from the group consisting of 3,3′-bis(triethoxysilylpropyl) disulfide, 3,3′-bis(trimethoxysilylpropyl) disulfide, 3,3′-bis(tributoxysilyl-propyl) disulfide, 3,3′-bis(tri-m-butoxysilyl-propyl) disulfide, 3,3′-bis(tripropoxypropyl) disulfide, 3,3′-bis(trihexoxysilylpropyl) disulfide, 2,2′-bis (dimethylmethoxysilylethyl) disulfide, 3,3′-bis(diphenylcyclohexoxysilylpropyl) disulfide, 3,3′-bis(ethyl-di-sec-butoxysilylpropyl) disulfide, 3,3′-bis(propyldiethoxysilylpropyl) disulfide, 3,3′-bis(triisopropoxysilylpropyl) disulfide, 12,12′-bis(triisopropoxysilylpropyl) disulfide, 3,3′-bis(dimethoxyphenylsilyl-2-methylpropyl) disulfide, and mixtures thereof.

In certain embodiments of the first and second embodiments disclosed herein, when the functionalizing compound is a tetrasulfide alkoxysilane, it is selected from the group consisting of bis(3-triethoxysilylpropyl) tetrasulfide, bis(2-triethoxysilylethyl) tetrasulfide, bis(3-trimethoxysilylpropyl) tetrasulfide, 3-trimethoxysilylpropyl-N,N-dimethylthiocarbamoyl tetrasulfide, 3-triethoxysilylpropyl-N,N-dimethylthiocarbamoyl tetrasulfide, 2-triethoxysilyl-N,N-dimethylthiocarbamoyl tetrasulfide, 3-trimethoxysilylpropyl-benzothiazole tetrasulfide, 3-triethoxysilylpropylbenzothiazole tetrasulfide, and mixtures thereof.

In certain embodiments of the first and second embodiments disclosed herein, the functionalizing compound is a mercaptosilane compound. Mercapto silane compounds can be described as having the general formula HS—R³—Si(Xₙ)(R⁴₃₋ₙ) where each X is independently selected from a halogen or an alkoxy group (if an alkoxy group, of the formula OR⁵ where R⁵ is a C₁ to C₆ aliphatic, cycloaliphatic or aromatic group); R³ is selected from a C₁ to C₄ alkylene; each R⁴ is independently selected from a C₁ to C₃₀ alkyl, C₇ to C₃₀ alkaryl, C₅ to C₃₀ cycloaliphatic or C₆ to C₂₀ aromatic; and n is an integer from 1 to 4. When X is a halogen, it can be selected from the group consisting of chlorine, bromine, iodine and fluorine, preferably chlorine. In certain preferred embodiments of the first and second embodiments disclosed herein, the functionalizing compound is a mercaptosilane having the above formula and R³ is selected from a C₁ to C₃ alkylene, X is an alkoxy group (with carbon portion of C₁ to C₆), and n is 3. 1-mercaptomethyltriethoxysilane, 2-mercaptoethyltriethoxysilane, 3-mercaptopropyltriethoxysilane, 3-mercaptopropylmethyldiethoxysilane, 2-mercaptoethyltriproxysilane, 18-mercaptooctadecyldiethoxychlorosilane

In certain embodiments of the first and second embodiments disclosed herein, the functionalizing compound is a blocked mercapto silane. Blocked mercapto silanes can be described as having the general formula B—S—R⁶—Si—X₃ with a blocking group B that replaces the mercapto hydrogen atom to “block” the reaction of the sulfur atom with the polymer. In certain embodiments of the first and second embodiments, where the blocked mercaptosilane has the foregoing general formula, B is a blocking group which can be in the form of an unsaturated heteroatom or carbon bound directly to sulfur via a single bond; R⁶ is selected from a C₁ to C₆ linear or branched alkyl chain, and each X is independently selected from the group consisting of C₁ to C₆ alkyl, C₁ to C₆ alkoxy, halogen, halogen-containing C₁ to C₆ alkyl, and halogen-containing C₁ to C₆ alkoxy. Suitable blocked mercapto silanes for use as the functionalizing compound in certain embodiments of the first and second embodiments disclosed herein, include, but are not limited to, those described in U.S. Pat. Nos. 6,127,468; 6,204,339; 6,528,673; 6,635,700; 6,649,684; 6,683,135; and 7,256,231. In certain embodiments of the first and second embodiments disclosed herein, when the functionalizing compound is a blocked mercapto silane it is selected from the group consisting of 2-triethoxysilyl-1-ethylthioacetate; 2-trimethoxysilyl-1-ethylthioacetate; 2-(methyldimethoxysilyl)-1-ethylthioacetate; 3-trimethoxysilyl-1-propylthioacetate; triethoxysilylmethyl-thioacetate; trimethoxysilylmethylthioacetate; triisopropoxysilylmethylthioacetate; methyldiethoxysilylmethylthioacetate; methyldimethoxysilylmethylthioacetate; methyldiiso-propoxysilylmethylthioacetate; dimethylethoxysilylmethylthioacetate; dimethylmethox-ysilylmethylthioacetate; dimethylisopropoxysilylmethylthioacetate; 2-triisopropoxysilyl-1-ethylthioacetate; 2-(methyldiethoxysilyl)-1-ethylthioacetate, 2-(methyldiisopropoxysilyl)-1-ethylthioacetate; 2-(dimethylethoxysilyl-1-ethylthioacetate; 2-(dimethylmethoxysilyl)-1-ethylthioacetate; 2-(dimethylisopropoxysilyl)-1-ethylthioacetate; 3-triethoxysilyl-1-propyl-thioacetate; 3-triisopropoxysilyl-1-propylthioacetate; 3-methyldiethoxysilyl-1-propyl-thioacetate; 3-methyldimethoxysilyl-1-propylthioacetate; 3-methyldiisopropoxysilyl-1-propylthioacetate; 1-(2-triethoxysilyl-1-ethyl)-4-thioacetylcyclohexane; 1-(2-triethoxysilyl-1-ethyl)-3-thioacetylcyclohexane; 2-triethoxysilyl-5-thioacetylnorbornene; 2-triethoxysilyl-4-thioacetylnorbornene; 2-(2-triethoxysilyl-1-ethyl)-5-thioacetylnorbornene; 2-(2-triethoxy-silyl-1-ethyl)-4-thioacetylnorbornene; 1-(1-oxo-2-thia-5-triethoxysilylphenyl)benzoic acid; 6-triethoxysilyl-1-hexylthioacetate; 1-triethoxysilyl-5-hexylthioacetate; 8-triethoxysilyl-1-octylthioacetate; 1-triethoxysilyl-7-octylthioacetate; 6-triethoxysilyl-1-hexylthioacetate; 1-triethoxysilyl-5-octylthioacetate; 8-trimethoxysilyl-1-octylthioacetate; 1-trimethoxysilyl-7-octylthioacetate; 10-triethoxysilyl-1-decylthioacetate; 1-triethoxysilyl-9-decylthioacetate; 1-triethoxysilyl-2-butylthioacetate; 1-triethoxysilyl-3-butylthioacetate; 1-triethoxysilyl-3-methyl-2-butylthioacetate; 1-triethoxysilyl-3-methyl-3-butylthioacetate; 3-trimethoxysilyl-1-propylthiooctanoate; 3-triethoxysilyl-1-propyl-1-propylthiopalmitate; 3-triethoxysilyl-1-propylthiooctanoate; 3-triethoxysilyl-1-propylthiobenzoate; 3-triethoxysilyl-1-propylthio-2-ethylhexanoate; 3-methyldiacetoxysilyl-1-propylthioacetate; 3-triacetoxysilyl-1-propylthioacetate; 2-methyldiacetoxysilyl-1-ethylthioacetate; 2-triacetoxysilyl-1-ethylthioacetate; 1-methyldiacetoxysilyl-1-ethylthioacetate; 1-triacetoxysilyl-1-ethylthioacetate; tris-(3-triethoxysilyl-1-propyl)trithiophosphate; bis-(3-triethoxysilyl-1-propyl)methyldithiophosphonate; bis-(3-triethoxysilyl-1-propyl)ethyldithiophosphonate; 3-triethoxysilyl-1-propyldimethylthiophosphinate; 3-triethoxysilyl-1-propyldiethylthiophosphinate; tris-(3-triethoxysilyl-1-propyl)tetrathiophosphate; bis-(3-triethoxysilyl-lpropyl)methyltrithiophosphonate; bis-(3-triethoxysilyl-1-propyl)ethyltrithiophosphonate; 3-triethoxysilyl-1-propyldi methyldithiophosphinate; 3-triethoxysilyl-1-propyldiethyldithiophosphinate; tris-(3-methyldimethoxysilyl-1-propyl)trithiophosphate; bis-(3-methyl-dimethoxysilyl-1-propyl)methyldithiophosphonate; bis-(3-methyldimethoxysilyl-1-propyl)-ethyldithiophosphonate; 3-methyldimethoxysilyl-1-propyldimethylthiophosphinate; 3-methyldi methoxysilyl-1-propyldiethylthiophosphinate; 3-triethoxysilyl-1-propyl methyl-thiosulfate; 3-triethoxysilyl-1-propylmethanethiosulfonate; 3-triethoxysilyl-1-propyl-etha nethiosulfonate; 3-triethoxysilyl-1-propylbenzenethiosulfonate; 3-triethoxysilyl-1-propyltoluenethiosulfonate; 3-triethoxysilyl-1-propylnaphthalenethiosulfonate; 3-triethoxysilyl-1-propylxylenethiosulfonate; triethoxysilylmethylmethylthiosulfate; triethoxysilyl methylmethanethiosulfonate; triethoxysilylmethylethanethiosulfonate; triethoxysilylmethylbenzenethiosulfonate; triethoxysilylmethyltoluenethiosulfonate; triethoxysilylmethylnaphthalenethiosulfonate; triethoxysilylmethylxylenethiosulfonate, and the like.

Processes for Preparing a Modified Guayule Resin Product

As discussed above, the second embodiment disclosed herein is directed to a process for preparing a modified guayule resin product. Generally, according to the process of the second embodiment, a guayule resin component is provided which comprises a mixture of argentatins having —OH groups, the guayule resin component can be considered to be a starting product. The —OH groups of the argentatins should be understood as an inherent part of the argentatin structure and provide a site for bonding to functionalizing compound, as discussed further infra. According to the process of the second embodiment, the guayule resin component is mixed with a functionalizing compound selected from alkoxysilanes, mercaptosilanes, and blocked mercaptosilanes to produce a modified guayule resin product comprising functionalized argentatins with at least one functional group provided by the functionalizing compound.

The amount of functionalizing compound that is used to prepare the modified guayule resin product (or more specifically the functionalized argentatins therein) may vary according to the process of the second embodiment. Generally, the amount of functionalizing compound can be calculated as a molar equivalent ratio of alkoxygroups from the functionalizing compound to —OH groups in the mixture of argentatins. Thus, use of an alkoxysilane compound having relatively more alkoxy groups (e.g., 6) can require the use of fewer moles of that alkoxysilane compound as compared to an alkoxysilane compound having only two alkoxy groups to produce the equivalent functionalization percentage of argentatins. In certain preferred embodiments of the second embodiment, the functionalizing compound is used in an amount sufficient to provide a molar equivalent ratio of alkoxy groups from the functionalizing compound to —OH groups in the mixture of argentatins of 1/1 to 6/1 (e.g., 1/1, 1.5/1, 2/1, 2.5/1, 3/1, 3.5/1, 4/1, 4.5/1, 5/1, 5.5/1 or 6/1), more preferably of 2/1 to 3/1 (e.g., 2.1/1, 2.2/1, 2.3/1, 2.4/1, 2.5/1, 2.6/1, 2.7/1, 2.8/1, 2.9/1 or 3/1).

In certain preferred embodiments of the process of the second embodiment, the use of an acid can be helpful to catalyze the reaction between the functionalizing compound and argentatin compounds. According to such embodiments, the amount of acid that is used may vary. In certain preferred embodiments of the process of the second embodiment, an acid is used in an amount sufficient to achieve a pH of 1 to 5 (e.g., 1, 1.5, 2, 2.5, 3, 3.5, 4, 4.5, or 5) in the mixture of the functionalizing compound and guayule resin component. Various acids may be used including, but not limited, to mineral acids. Exemplary mineral acids include hydrochloric acid, sulfuric acid and nitric acid.

In certain preferred embodiments of the process of the second embodiment, heat is applied after mixing the guayule resin component with the functionalizing compound. In those embodiments where an acid is added to catalyze the reaction between the functionalizing compound and the argentatins of the guayule resin component, any heat that is applied is preferably applied after the acid has been added. In those embodiments of the second embodiment where heat is applied, the temperature to which the mixture (i.e., of functionalizing compound, guayule resin component, and optionally acid) is heated may vary; in preferred embodiments, the mixture is heated to a temperature of 20 to 100° C. (e.g., 20, 25, 30, 35, 40, 45, 50, 55, 60, 65, 70, 75, 80, 85, 90, 95 or 100° C.).

As those of skill in the art will appreciate, the reaction between the functionalizing compound and the argentatins of the guayule resin component will produce by-product alcohol. Thus, in certain embodiments of the process of the second embodiments, the process further comprises removing the by-product alcohol from the modified guayule resin product. Various processes may be used for removing the by-product alcohol, including heat and/or vacuum.

### Working Examples

The following examples illustrate specific and exemplary embodiments and/or features of the embodiments of the present disclosure. The examples are provided solely for the purposes of illustration and should not be construed as limitations of the present disclosure. Numerous variations over these specific examples are possible without departing from the spirit and scope of the presently disclosed embodiments. It should specifically be understood that modified guayule resin products can be prepared using different amounts of guayule resin component (containing different amounts of argentatins), as discussed above, and using different amounts or types of functionalizing compounds, also as discussed above.

Example 1 (production of a modified guayule resin product): A portion of guayule resin (a guayule resin component) was mixed with a functionalizing compound in the form of n-octadecyltrimethoxysilane (97% purity, obtained from Gelest in liquid form, molecular weight 374.7 grams/mole). The guayule resin component contained about 80 weight % argentatins and 3.8 weight % low molecular weight rubber (determined by low molecular weight GPC). A 1 gram portion of the functionalizing compound was added to a 2 gram portion of the guayule resin component, then 1 milliliter of water was added to promote the condensation reaction. The mixture was heated to boiling on a hot plate for about 1 hour and then placed in a vacuum oven at 75° C. for 2 hours to remove any evolved methanol. Table 1 below shows the change in properties of the modified guayule resin product as compared to those of the guayule resin component, which change is believed to be due to the reaction of the argentatins in the guayule resin component with the functionalizing compound causing the methyl groups to be replaced by argentatins. As can be seen from the data in Table 1, the Mn, Mw and Mz values for the modified guayule resin product are increased as compared to the corresponding values for the guayule resin component in unmodified form. Table 1 also shows the percentage by weight of different components of the guayule resin component or modified guayule resin product (calculated from the area under the peak for selected peaks from the GPC. Component 1 corresponds to oligomerized product (first peak), component 2 corresponds to resin triglycerides and lower molecular weight oligomerized product (from peaks 2 and 3, ranging from an Mn of 850 to 2000 grams/mole) and component 3 corresponds to unmodified resin in the form of argentatins and guayules (from peaks 4 and 5, ranging from an Mn of 400 to 850 grams/mole). According to the data in Table 1, the modified guayule resin product of Example 1 has a lesser amount (weight percentage) of argentatins (as evidenced by a lower percentage of component 3) and a higher amount of oligomerized material (as evidenced by a higher percentage of component 1, which corresponds to the product produced from the reaction of the argentatins in the guayule resin component with the alkoxysilane). The Tg was determined by DSC and the molecular weight values were measured using GPC (with a TOSOH column, THF as the eluting solvent and a polystyrene standard).

TABLE 1

| | | Mn | Mw | Mz | | | |
|---|---|---|---|---|---|---|---|
| | | (grams/ | (grams/ | (grams/ | Component | Component | Component |
| | Tg ° C. | mole) | mole) | mole) | 1 | 2 | 3 |
| Guayule | 38.54 | 1244 | 1588 | 2568 | 3.8% | 16.0% | 80.2% |
| resin | | | | | | | |
| component | | | | | | | |
| Example 1 | 1.97 | 3551 | 4646 | 7064 | 30.3% | 21.3% | 48.4% |

Examples 2-5 (production of modified guayule resin products): A portion of guayule resin (a guayule resin component) was mixed with one of four functionalizing compounds. For Example 2, polydimethoxysilane was utilized (obtained from Gelest, under product name PSI-026). For Example 3, n-propyltrimethoxysilane was utilized (obtained from Gelest, under product name SIP 6918.0). For Example 4, 3-mercaptopropyltrimethoxysilane was utilized (obtained from Gelest, under product name SIM 6474.0). For Example 5, 4-tetraethoxysilane was utilized (obtained from Gelest, under product name SIT 7110.0). Each of the functionalizing compounds was present in the form of a liquid. The guayule resin component (which was different than the guayule resin component used in Example 1) was present in toluene solution with the primary component of the guayule resin component being a mixture of argentatins. The guayule resin component contained about 76% by weight argentatins and 0.0 weight % rubber (determined by low molecular weight GPC). For each of the examples, the respective functionalizing compound was added to a portion of the guayule resin component in toluene solution in the amount indicated below in Table 2. After mixture of the guayule resin component with the respective functionalizing compound, 2 milliliters of an acidic ethanol-water solution was added to each mixture and then placed on a hot plate for a few hours (without stirring). (The acidic ethanol-water solution was prepared by making 10 milliliters of a 10% solution of water and ethanol and adding 1 milliliter of 6 N hydrochloric acid.) The hot plate procedure took place on a Friday and the samples were allowed to rest (at room temperature) over the weekend. On Monday morning each sample was placed in a vacuum oven at 100° C. for 4 hours and then sent for testing. Table 3 below shows the change in properties of the modified guayule resin product as compared to those of the guayule resin component, which change is believed to be due to the reaction of the argentatins in the guayule resin component with the functionalizing compound causing the methyl or ethyl groups to be replaced by argentatins. Table 3 also shows the percentage by weight of different components of the guayule resin component or modified guayule resin product (calculated from the area under the peak for selected peaks from the GPC. Component 1 corresponds to oligomerized product (first peak), component 2 corresponds to resin triglycerides and lower molecular weight oligomerized product (from peaks 2 and 3, ranging from an Mn of 850 to 2000 grams/mole) and component 3 corresponds to unmodified resin in the form of argentatins and guayules (from peaks 4, 5 and 6, ranging from an Mn of 400 to 850 grams/mole). According to the data in Table 3, each of the modified guayule resin products in Example 2-5 has a lesser amount (weight percentage) of argentatins (as evidenced by a lower percentage of component 3) and a higher amount of oligomerized material (as evidenced by a higher percentage of component 1, which corresponds to the product produced from the reaction of the argentatins in the guayule resin component with the functionalizing compound). Although only Example 4 shows an increase in Mn, Mw and Mz, the fact that percentage of component 3 decreases for each of Examples 2-5 and the amount of component 1 increases for each of Example 2-5 is an indication that a reaction has occurred between the argentatins in the guayule resin component and the functionalizing compound. The Tg was determined by DSC and the molecular weight values were measured using GPC (with a TSOH column, THF as eluting solvent and a polystyrene standard).

TABLE 2

| | Amount of guayule resin | Amount of functionalizing |
|---|---|---|
| | component (grams) | compound (grams) |
| Example 2 | 4.91 | 1.23 |
| Example 3 | 4.95 | 0.85 |
| Example 4 | 5.00 | 1.08 |
| Example 5 | 4.98 | 0.66 |

TABLE 3

| | | Mn | Mw | Mz | Component | | |
|---|---|---|---|---|---|---|---|
| | | (grams/ | (grams/ | (grams/ | 1 (rubber/ | Component | Component |
| | Tg ° C. | mole) | mole) | mole) | oligomer) | 2 | 3 |
| Guayule | −68.14 | 1660 | 4075 | 18,759 | 5.7% | 30.8% | 63.5% |
| resin | | | | | | | |
| component | | | | | | | |
| Example 2 | −3.82 | 1625 | 3834 | 16,499 | 5.8% | 33.6% | 60.6% |
| Example 3 | 3.6 | 1873 | 3996 | 11,779 | 10.6% | 42.5% | 47.0% |
| Example 4 | 58.47 | 2327 | 9622 | 36,472 | 32.8% | 20.4% | 46.8% |
| Example 5 | −10.1 | 1598 | 3592 | 14,906 | 5.3% | 33.1% | 61.7% |

This application discloses several numerical range limitations that support any range within the disclosed numerical ranges, even though a precise range limitation is not stated verbatim in the specification, because the embodiments of the compositions and methods disclosed herein could be practiced throughout the disclosed numerical ranges. With respect to the use of substantially any plural or singular terms herein, those having skill in the art can translate from the plural to the singular or from the singular to the plural as is appropriate to the context or application. The various singular or plural permutations may be expressly set forth herein for sake of clarity.

It will be understood by those within the art that, in general, terms used herein, and especially in the appended claims are generally intended as “open” terms. For example, the term “including” should be interpreted as “including but not limited to,” the term “having” should be interpreted as “having at least,” the term “includes” should be interpreted as “includes but is not limited to.” It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” All references, including but not limited to patents, patent applications, and non-patent literature are hereby incorporated by reference herein in their entirety. While various aspects and embodiments of the compositions and methods have been disclosed herein, other aspects and embodiments will be apparent to those skilled in the art. The various aspects and embodiments disclosed herein are for purposes of illustration and are not intended to be limiting, with the true scope and spirit being indicated by the claims.

What is claimed is:

1-25. (canceled)

26. A modified guayule resin product comprising a mixture of argentatins having at least one functional group, wherein the at least one functional group is provided by a functionalizing compound selected from alkoxysilanes, mercaptosilanes, and blocked mercaptosilanes.

27. The modified guayule resin product of claim 26, wherein the functionalizing compound is a sulfur-containing alkoxysilane.

28. The modified guayule resin product of claim 27, wherein the functionalizing compound is a disulfide alkoxysilane compound selected from the group consisting of 3,3′-bis(triethoxysilylpropyl) disulfide, 3,3′-bis(trimethoxysilylpropyl) disulfide, 3,3′-bis(tributoxysilyl-propyl) disulfide, 3,3′-bis(tri-m-butoxysilyl-propyl) disulfide, 3,3′-bis(tripropoxypropyl) disulfide, 3,3 ‘-bis(trihexoxysilylpropyl) disulfide, 2,2’-bis(dimethylmethoxysilylethyl) disulfide, 3,3 ‘-bis(diphenylcyclohexoxysilylpropyl) disulfide, 3,3’-bis(ethyl-di-sec-butoxysilylpropyl) disulfide, 3,3 ‘-bis(propyldiethoxysilylpropyl) disulfide, 3,3’-bis(triisopropoxysilylpropyl) disulfide, 12, 12′-bis(triisopropoxysilylpropyl) disulfide, 3,3′-bis(dimethoxyphenylsilyl-2-methylpropyl) disulfide, and combinations thereof.

29. The modified guayule resin product of claim 26, wherein the functionalizing compound is a non-sulfur containing alkoxysilane selected from
(a) non-sulfur containing alkoxysilanes having formula (I):
R¹₍₄₋ₙ₎−Si(OR²)ₙ (I)
where n=2, 3, or 4; each R¹ is independently selected from hydrocarbyl groups having 1-24 carbons; and R² is an alkyl group having 1-10 carbons, or
(b) non-sulfur containing bis-alkoxysilanes having 4 to 6 alkoxygroups.

30. The modified guayule resin product of claim 29, wherein the functionalizing compound has formula (I) and n=3, R¹ is selected from alkyl groups having 6-24 carbons, and R² is methyl or ethyl.

31. The modified guayule resin product of claim 26, wherein the functionalizing compound is a mercaptosilane or a blocked mercaptosilane.

32. The modified guayule resin product of claim 26, having a Tg of −70 to 100° C.

33. The modified guayule resin product of claim 32, having a Tg of 1 to 100° C.

34. The modified guayule resin product of claim 26, wherein the modified guayule resin has a Mn of 1600 to 5000 grams/mole and/or a Mw of 2000 to 10000 grams/mole.

35. The modified guayule resin product according to claim 26, wherein the mixture of argentatins have at least 50% by mass of hydrogen atoms from their —OH groups replaced with the at least one functional group.

36. The modified guayule resin product according to claim 26, wherein less than 10% by weight is present in the modified guayule resin product.

37. The modified guayule resin product according to claim 26, wherein the mixture of functionalized argentatins constitutes at least 40% by weight of the modified guayule resin product.

38. The modified guayule resin product according to claim 26, wherein the mixture of modified argentatins is a primary component by weight of the modified guayule resin product.

39. A process for preparing a modified guayule resin product comprising
a. providing a guayule resin component which comprises a mixture of argentatins having —OH groups,
b. mixing the guayule resin component with a functionalizing compound selected from alkoxysilanes, mercaptosilanes, and blocked mercaptosilanes, and
c. producing a modified guayule resin product comprising functionalized argentatins with at least one functional group provided by the functionalizing compound.

40. The process of claim 39, wherein the mixture of argentatins is a primary component by weight of the guayule resin component.

41. The process of claim 39, wherein the mixture of argentatins constitutes at least 40% by weight of the guayule resin component.

42. The process of claim 39, wherein the functionalizing compound is used in an amount sufficient to provide a molar equivalent ratio of alkoxy groups from the functionalizing compound to —OH groups in the mixture of argentatins of 1/1 to 6/1.

43. The process of claim 39, wherein (b) includes adding an acid.

44. The process of claim 39, wherein heat is applied after mixing the guayule resin component with a functionalizing compound.

45. The process of claim 39, wherein (c) includes production of by-product alcohol generated from the alkoxy group of the alkoxysilane, and the process further comprises removing the by-product alcohol.
